(19) United States
(12) Patent Application Publication
(10) Pub. No.: US 2025/0325771 A1
(43) Pub. Date: Oct. 23, 2025

(54) AUTO IDENTIFICATION OF PATIENT INTERFACES BY MEASUREMENT OF SIGNATURE FORCES AND/OR VIBRATIONS AT MASK CONNECTIONS

(71) Applicant: KONINKLIJKE PHILIPS N.V., EINDHOVEN (NL)

(72) Inventors: DAAN ANTON VAN DEN ENDE, BREDA (NL); JOYCE VAN ZANTEN, WAALRE (NL); JONATHAN SAYER GRASHOW, CHESWICK, PA (US); CORNELIS PETRUS HENDRIKS, EINDHOVEN (NL); JOACHIM JOHANNES KAHLERT, AACHEN (DE); ELIZABETH POWELL MARGARIA, SEWICKLEY, PA (US)

(21) Appl. No.: 19/182,714

(22) Filed: Apr. 18, 2025

Related U.S. Application Data
(60) Provisional application No. 63/636,918, filed on Apr. 22, 2024.

(51) Int. Cl. A61M 16/06; A61M 16/00

(52) U.S. Cl. CPC A61M 16/0683; A61M 16/0003; A61M 16/024; A61M 2202/0007; A61M 2202/02; A61M 2205/0216; A61M 2205/332; A61M 2205/3327; A61M 2205/3331; A61M 2205/50; A61M 2205/60; A61M 2205/6054; A61M 2210/06

(57) ABSTRACT

A system and method for automatically identifying a mask used in a pressure support system. An output of an identification sensor arrangement coupled to a patient interface device is received in a controller. The identification sensor arrangement includes: (i) a force sensor structured and configured to generate force data indicative of a force generated during connection of a mask portion to a headgear portion or to a conduit portion of the patient interface device or during use of the pressure support system, and/or (ii) a vibration sensor structured and configured to generate vibration data indicative of vibrations generated during connection of the mask portion to the headgear portion or to the conduit portion or during use of the pressure support system. The controller determines the brand, type and/or size of the mask portion based on the force data and/or the vibration data.

### CROSS-REFERENCE TO RELATED APPLICATIONS

This application claims priority under 35 U.S.C. § 119(e) from provisional U.S. patent application No. 63/636,918, filed Apr. 22, 2004, the contents of which are incorporated herein by reference.

### BACKGROUND OF THE INVENTION

### 1. Field of the Invention

The disclosed concept relates generally to pressure support systems, and, more particularly, to a pressure support system in which force and/or vibration information are utilized to automatically identify the patient interface device being used in the system.

### 2. Description of the Related Art

Today, the first line therapy for patients diagnosed with obstructive sleep apnea syndrome (OSAS) after a sleep test is pressure assisted ventilation support, also known as PAP support or therapy, most often by continuous positive airway pressure (CPAP) therapy. Such pressure assisted ventilation support involves the placement of a respiratory patient interface device, including a mask component, on the face of a patient. The mask component may be, for example and without limitation, a nasal mask that covers the patient's nose, a nasal cushion having nasal prongs that are received within the patient's nares, a pillow-style nasal cushion that engages the patient's nares without being inserted therein, a nasal/oral mask that covers the patient's nose and mouth, or a full-face mask that covers the patient's face. The respiratory patient interface device interfaces a pressure/flow generating device (also known as a PAP device) with the airway of the patient so that a flow of breathing gas can be delivered from the pressure/flow generating device to the airway of the patient.

In moderate and severe OSAS patients with an apnea-hypopnea index (AHI) that is greater than 15, the therapy is reimbursed. In mild OSAS patients with daytime symptoms, or with chronic and persistent cardiac comorbidities, the PAP therapy is reimbursed for an AHI that is greater than 5. Reimbursement covers the PAP device as well as periodic resupply of consumable items, such as tubing, headgear, masks, and cushions. Different time periods for replacement of these consumable items are in effect, mostly ranging from 1 month to 6 months.

The proper setup of the PAP device including, for instance, pressure settings and fitting of the mask, is done by a qualified sleep clinician, most often in an overnight setting at a sleep lab, although home titration is a possible alternative for certain patients. Once the correct machine settings and appropriate consumable items have been established, the equipment is supplied by a durable medical equipment (DME) supplier and the patient commences therapy.

When the mask is due for replacement, the DME may supply a new mask to the patient. Often, DMEs send their patients not just one mask but a pack that contains all available mask sizes (a so-called “fit pack”), since it is easier for DMEs to send such a pack which contains all mask sizes when they do not know exactly the mask size that the patient is using. Patients can use any of the masks in this pack after receiving it and may intentionally or accidentally switch mask sizes without the clinician or DME knowing which mask they are using. Also, patients or DMEs may replace the mask with a different type of mask than was originally prescribed. In some cases. patients might even switch mask brands without the clinician or DME knowing.

The identification of the actual mask type, size and/or brand that is used during therapy at any time is a need that is expressed by clinicians and DMEs alike. This would allow clinicians and/or DMEs to improve the understanding of collected CPAP data and diagnose problems more accurately (especially in a remote/telehealth setting), to manage resupply, reimbursement, and product lifetime, and to remotely control device settings depending on the type of mask that is used.

### SUMMARY OF THE INVENTION

In one embodiment, a pressure support system is provided that includes a pressure generating device for generating a flow of breathing gas, a patient interface device for delivering the flow of breathing gas to the airways of a patient, wherein the patient interface device includes a mask portion, a headgear portion, and a conduit portion, the conduit portion being for receiving the flow of breathing gas from the pressure generating device, an identification sensor arrangement coupled to the patient interface device, wherein the identification sensor arrangement includes: (i) a force sensor structured and configured to generate force data indicative of a force generated during connection of the mask portion to the headgear portion or to the conduit portion or during use of the pressure support system, and/or (ii) a vibration sensor structured and configured to generate vibration data indicative of vibrations generated during connection of the mask portion to the headgear portion or to the conduit portion or during use of the pressure support system, and a controller structured and configured to receive an output of the identification sensor arrangement and determine a brand, type and/or size of the mask portion based on the force data and/or the vibration data.

In another embodiment, a method for automatically identifying a mask used in a pressure support system including a pressure generating device for generating a flow of breathing gas, a patient interface device for delivering the flow of breathing gas to the airways of a patient, wherein the patient interface device includes a mask portion, a headgear portion, and a conduit portion, the conduit portion being for receiving the flow of breathing gas from the pressure generating device. The method includes receiving in a controller an output of an identification sensor arrangement coupled to the patient interface device, wherein the identification sensor arrangement includes: (i) a force sensor structured and configured to generate force data indicative of a force generated during connection of the mask portion to the headgear portion or to the conduit portion or during use of the pressure support system, and/or (ii) a vibration sensor structured and configured to generate vibration data indicative of vibrations generated during connection of the mask portion to the headgear portion or to the conduit portion or during use of the pressure support system, and determining in the controller a brand, type and/or size of the mask portion based on the force data and/or the vibration data.

### BRIEF DESCRIPTION OF THE DRAWINGS

A full understanding of the invention can be gained from the following description of the preferred embodiments when read in conjunction with the accompanying drawings in which:

FIG. 1 is a schematic diagram of a pressure support system in which specific characteristic connecting forces are used to automatically identify the mask used in the system according to a non-limiting exemplary embodiment of the disclosed concept;

FIG. 2 is a schematic diagram of an electronics module forming part of the system of FIG. 1 according to an exemplary embodiment;

FIG. 3 is a schematic diagram of a patient interface configuration employing a snap-fit connection that may be implemented in connection with the system of FIG. 1 wherein force information is used to identify a component of the patient interface configuration;

FIG. 4 is a schematic diagram of an alternative patient interface configuration employing a magnet based connection that may be implemented in connection with the system of FIG. 1 wherein force information is used to identify a component of the patient interface configuration;

FIG. 5 is a schematic diagram of another alternative patient interface configuration employing a snap-fit connection that may be implemented in connection with the system of FIG. 1 wherein force information is used to identify a component of the patient interface configuration;

FIG. 6 illustrates force profiles associated with the patient interface configuration of FIG. 5;

FIG. 7 is a schematic diagram of another alternative patient interface configuration employing a snap-fit connection that may be implemented in connection with the system of FIG. 1 wherein force information is used to identify a component of the patient interface configuration;

FIG. 8 illustrates force profiles associated with the patient interface configuration of FIG. 7;

FIG. 9 illustrates force profiles associated with another patient interface configuration according to the disclosed concept;

FIG. 10 is a schematic diagram of yet another alternative patient interface configuration employing a snap-fit connection that may be implemented in connection with the system of FIG. 1 wherein force information is used to identify a component of the patient interface configuration;

FIG. 11 is a schematic diagram of another alternative patient interface configuration wherein force information is used to identify a component of the patient interface configuration;

FIG. 12 is a schematic diagram of mask clip arrangement employing a snap-fit connection that may be implemented in connection with the system of FIG. 1 wherein vibration information is used to identify a component of the patient interface configuration;

FIG. 13 is a schematic diagram of another mask clip arrangement employing a snap-fit connection that may be implemented in connection with the system of FIG. 1 wherein vibration information is used to identify a component of the patient interface configuration;

FIG. 14 is a schematic diagram of mask clip arrangement employing a magnetic connection that may be implemented in connection with the system of FIG. 1 wherein vibration information is used to identify a component of the patient interface configuration; and

FIG. 15 is a schematic diagram of mask clip arrangement employing a snap-fit connection and a bistable element that may be implemented in connection with the system of FIG. 1 wherein vibration information is used to identify a component of the patient interface configuration.

### DETAILED DESCRIPTION OF THE INVENTION

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, the term “number” shall mean one or an integer greater than one (i.e., a plurality).

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As noted elsewhere herein, the identification of the actual patient interface that is used during pressure support therapy at any time is a need that is expressed by clinicians and DMEs for trouble shooting problems with therapy and improving therapy adherence. In many patient interfaces, physical connections are used for attaching and detaching the different components of the patient interface, such as the mask cushion, tubing and/or headgear components. In some patient interfaces, the tubing is directly connected to the cushion. In others, the tubing and cushion are attached to the headgear. Patient interface straps are also often attached with clips to allow fast and easy attachment to enhance patient experience when taking a mask on and off.

The disclosed concept, as described in detail herein in connection with a number of exemplary embodiments, provides a novel way of identifying a type and/or size of a patient interface and/or a component thereof using characteristics of the mechanical connections made during use of the patient interface. The connection may include, for example and without limitation, connections between a mask and headgear or between a mask and a tube, depending on the patient interface design.

In one aspect, the mask type and/or size may be identified by measuring mask specific characteristic connecting forces statically or dynamically, such as by measuring peak force and/or derivatives of the force over time, which can be defined by the mechanical design of the connections. As a result, the type and/or size of a patient interface component such as a mask or cushion that is connected may be identified, and unidentified or incorrect masks may be noted and flagged to the patient or a caregiver.

The disclosed concept as just described may thus be implemented by an exemplary system that includes: (i) a patient interface connected to a PAP device by an air tube or the like wherein at least one physical connection to a component of the patient interface, such as a mask, is employed with specified connecting forces that are linked to the component type and size, (ii) a force sensor, (iii) a unit for sensor readout, (iv) a communication means for the data, (v) a controller that determines the type and/or size of the component based on the sensor value(s), and (vi) a power supply for the sensor, readout, communications and controller elements. In the exemplary embodiment, the force sensor is positioned at or near the physical connection so it can detect the specific connecting force properties of the specified physical connection.

In another aspect, the mask type and size may be identified by measuring the vibration profile when the patient makes a connection between the mask and tubing and/or straps or when the therapy device provides pressure to the mask. The vibration profile is a specific characteristic of each mask and is defined by the mechanical design of the mask and/or connecting element. As a result, the type and size of a patient interface component such as a mask may be identified, and unidentified or incorrect masks may be noted and flagged to the patient or a caregiver.

The disclosed concept as just described in this aspect may thus be implemented by an exemplary system that includes: (i) a patient interface connected to a PAP device by an air tube containing at least one connection to the patient interface, (ii) at least one element with a specified geometry that causes a characteristic vibration each time the element is activated (for example, the element can be a physical connection between two parts of the mask), (iii) a vibration sensor located close to the physical connection, (iv) a unit for sensor readout, (v) a communication means for the data, (vi) a controller that compares the sensor readout to a database of vibration profiles in order to determine the type and/or size of the mask, and (vii) a power supply for the sensor, readout, communications and controller elements. The sensor is positioned at or near the physical connection so it can detect the specific vibration properties of the specified physical connection so that the mask can be identified by the controller when a connection is made. In this aspect, the mask is identified using the following procedure: (i) initiating event (e.g., attachment of mask/cushion or start of therapy), (ii) readout of vibration sensor, (iii) analysis of vibration data (e.g., amplitude/frequency/decay), (iv) comparison of measured vibration characteristic using, for example, a lookup table for masks, (v) identification of mask (stored as the mask in use), and (vi) message patient/caregiver if identified mask is different than previous mask in use.

### Force-Based Embodiments

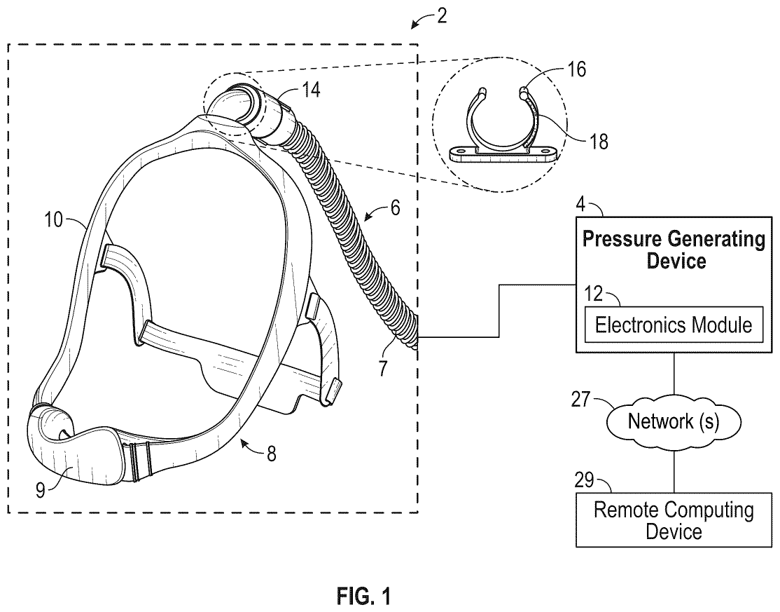

FIG. 1 is a schematic diagram of a pressure support system 2 in which specific characteristic connecting forces are used to automatically identify the mask used in the system according to a non-limiting exemplary embodiment of the disclosed concept. In this embodiment, the connection force applied to a clip is used to identify the body that the clip is connected to. The force sensor can be integrated in the clip or located between the clip and the connecting body. The force sensor may employ any kind of force sensing mechanism, such as resistive, capacitive or optical sensors. The clip can be embodied in many ways, one particular example of which is shown in FIG. 1 and described in detail below.

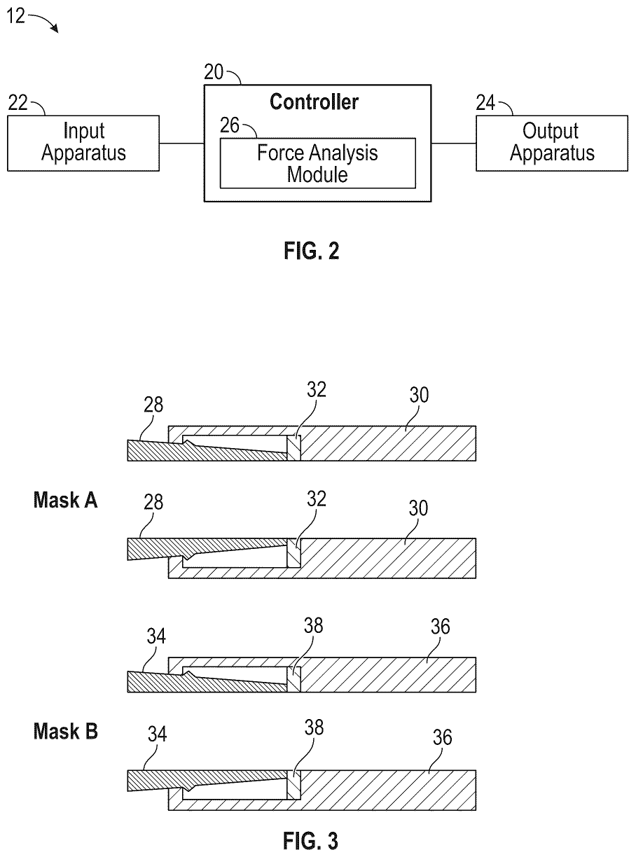

Referring to FIG. 1, pressure support system 2 is adapted to provide a regimen of respiratory therapy to a patient. Pressure support system 2 includes a pressure generating device 4 (also known as a PAP device) and a patient circuit 6 including a delivery conduit 7 and a patient interface device 8. In the illustrated embodiment, patient interface device 8 includes a mask 9 and a headgear 10 for securing mask 9 to the head of the patient. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Koninklijke Philips N.V.), and auto-titration pressure support devices. As seen in FIG. 1, pressure generating device 4 includes an electronics module 12, which is described in greater detail herein in connection with FIG. 2.

Delivery conduit 7 is structured to communicate the flow of breathing gas from pressure generating device 4 to patient interface device 8. Typically, delivery conduit 7 includes one or more individual conduits or tubes, a first end of which couples with pressure generating device 4 and a second end of which couples with patient interface device 8. In the illustrated embodiment, the second end is coupled with patient interface device 8 through a fluid coupling device 14 of patient interface device 8.

In the exemplary embodiment illustrated in FIG. 1, mask 9 is a nasal cushion structured to be placed the nares of a patient. Any type of mask, however, which facilitates the delivery of the flow of breathing gas to, and the removal of a flow of exhalation gas from, the airway of such a patient may be used while remaining within the scope of the disclosed concept. An opening in the top of headgear 10, to which fluid coupling device 14 is coupled by way of a clip 16, allows the flow of breathing gas from pressure generating device 4 to be communicated to an interior space defined by headgear 10 and mask 9, and then to the airway of the patient. In the illustrated exemplary embodiment, clip 16 is fixed to the top of headgear 10 and allows for the quick connection of delivery conduit 7 to and quick release of delivery conduit 7 from headgear 10.

As seen in FIG. 1, clip 16 includes a force sensor 18, such as a strain gage, to monitor the clamping force of clip 16 on delivery conduit 7 when delivery conduit 7 is clicked in. The force signals captured by force sensor 18 may be provided to electronics module 12 described below using a wired or wireless connection so that electronics module 12 is able determine the type and/or size of the component of patient interface device 8 based on the sensor value(s). For example, if delivery conduit 7 has a standard connector size (which is needed to fit the quick release), the force sensing clip 16 may be a slightly different size for each mask. These slight differences will result in a variation in clamping force that is exerted by clip 16 on delivery conduit 7, with each specific patient interface device 8 having a corresponding force range to be able to identify the type of mask or other component thereof.

FIG. 2 is a block diagram of electronics module 12 according to an exemplary embodiment of the disclosed concept. Electronics module 12 includes a controller 20, an input apparatus 22 (such as a keyboard), and an output apparatus 24 (such as a liquid crystal display). A user is able to provide input into controller 20 using input apparatus 22, and controller 20 provides output signals to output apparatus 24 to enable output apparatus 24 to display information to the user. Controller 20 implements a force analysis module 26 that is configured to determine the type and/or size of the component based on the sensor value(s) received from force sensor 18. In the exemplary embodiment, force analysis module 26 is implemented by a number routines that are executable by the processor portion of controller 20. In one embodiment, the type and/or size of the component of patient interface device 8 may be determined based on the sensor value(s) received from force sensor 18 using a comparison algorithm implemented in force analysis module that is based on a lookup table or database of force values and corresponding patient interface device component types and sizes. In an alternative embodiment, the type and/or size of the component of patient interface device 8 may be determined based on the sensor value(s) received from force sensor 18 using a trained machine learning system, such as a trained convolutional neural network (CNN), implemented by force analysis module 26.

The force profile collected by force sensor 16 resulting from making connections as described herein will depend on the particulars of the structure and materials of the components of patient interface device 8. The trained machine learning system of the exemplary embodiment can be trained to classify the type, size and/or brand of the components of patient interface device 8 using force data collected in sleep labs or during home sleep tests (using patient populations with different masks and device settings). The trained machine learning system may also be improved with new incoming data (while patients are using system 2). Ground truth for the training may be provided by limiting the patient use data to data from nights that are verified for the correct mask (for instance by asking whether the patient is still using the original mask) and/or by using data from only the nights before a follow up lab visit or DME telephone follow up or mobile application chat where the patient is asked by staff which mask they used. This limits the incoming data but is advantageous to preserve quality. Alternatively, the learning algorithm may randomly ask patients to submit their mask ID (in form of QR code or other common way) to verify the force data with ground truth.

One challenge for the disclosed concept is the need to classify multiple brands and mask types and sizes. In one example, it may be necessary to classify 6 brands, 5 mask types, and 3 mask sizes, meaning a total of 90 classes or categories. An additional complication is the interaction between the patient and the mask and between the pressure and the mask. In general, the number of classes/categories a classifier can classify with good precision is determined by: (i) how distinct each category is, (ii) how many features can be derived from the content, and (iii) how many high-quality labeled examples are available. In principle, it is possible to build a trained machine learning system, such as a CNN, with 90 outputs (labels) or more. For example, in known car recognition applications using a CNN, 196 classes of cars have been able to be distinguished using a car data set consisting of 16,000 images (8000 for training and 8000 for testing).

A practical problem is how to get such an amount of training data for the disclosed concept. One option is a large study with thousands of patients. Another option is to gather data from clinical practice using force and/or vibration measurements during therapy set-up and/or use when the mask type and size is known. Alternatively, data from later-on in the therapy can be used when the mask has been changed (consciously and known by the patient and therapist). An option to improve the model is to ask questions such as “did you change your mask?”, or “are you now using mask X?” when a change in force is detected.

One way to quickly generate a large amount of training data is to use in silico data which can be generated with force-based simulations.

As seen in FIG. 1, in the disclosed concept, pressure generating device 4 is coupled to one or more communications networks 27. A remote computing device 29, such as a server computer or an edge device, is also coupled to the network(s) 27. In the exemplary embodiment, remote computing device 29 is a computerized sleep and respiratory care management system, such as the Care Orchestrator system provided by Koninklijke Philips N.V. Thus, the mask identification that is performed by force analysis module 26 as described herein can be communicated to remote computing device 29 so that information relating thereto can be provided to a caregiver or a DME. As noted elsewhere herein, that information may be in the form a warning (“mask changed”) or as a piece of information (mask type, size, and/or brand) that is used by the sleep and respiratory care management system.

Alternatively, the force sensor may be fully located in the tubing or in a separate add-on conduit that can be attached between the tubing and the mask of a patient interface device, for instance by a capacitive compression sensor which is compressed differently due to the specific geometry of the connection at the mask side. An advantage of this measurement is that the mask connector geometry only changes slightly for each type and does not result in major changes in manufacturing of each type of mask and the sensor is located in the tubing or in a separate conduit element that is not part of the (disposable) mask. An example of this alternative embodiment, which may be used in connection with pressure generating device 4 in system 2, is shown in FIG. 3 for differentiating between mask A and mask B. Specifically, in this embodiment, mask A includes a mask connector 28 structured to be coupled to a delivery conduit 30 (or a fluid coupling conduit), wherein delivery conduit 30 has force sensor 32 in the form of a capacitive compression sensor, mounted therein.

Mask B includes a mask connector 34 structured to be coupled to a delivery conduit 36 (or a fluid coupling conduit), wherein delivery conduit 36 has force sensor 38 in the form of a capacitive compression sensor, mounted therein. In this embodiment, the geometry of mask connector 28 and/or delivery conduit 30 is different from the geometry of mask connector 34 and/or delivery conduit 36 to allow them to be differentiated from one another based on the compressive forces measured by force sensors 32 and 38, respectively. Also, in this embodiment, pressure generating device 4 may be programmed to cease operation if force sensor 32, 38 is not activated at all, which could indicate lack of mask connection or an improper mask that is connected (for instance a competitor or old mask type that does not support this type of identification). Also, an alert can be generated by pressure generating device 4 to the patient to check their mask connection or fit the correct mask.

In a further alternative embodiment, the sensing of the clip to cushion connection forces according to the disclosed concept can be used to identify both the mask type and the cushion type. For instance, the magnetic field strength between two magnets in magnetic-type clips can be measured using force sensors positioned between the magnet-on-magnet connections, which are typically employed in such magnetic clips in masks. Since the force between two magnets is dependent on the remnant magnetization to the second power, a relatively small change in magnetization will lead to a discernible change in measured force. Alternatively, a spacer may be placed between the magnet-on-magnet connection on the mask side.

By controlling spacer thickness for different mask types and sizes, the force may be controlled so different masks can be identified. This has the advantage that the same magnets can be used for each mask, but the mask geometry (in the form of the spacer thickness) defines the attractive force by which the mask can be identified. Alternatively, a combination of two or more different variations may be employed, such as variations in thickness, magnetization, and size. Obviously, the connection strength will vary between masks which may be seen as a problem, even by the experts in the field. However, since the connection forces needed for the clips are relatively small compared to the maximum force that patients can typically supply to disconnect the clips, there is a range between the minimum and maximum connection force limits.

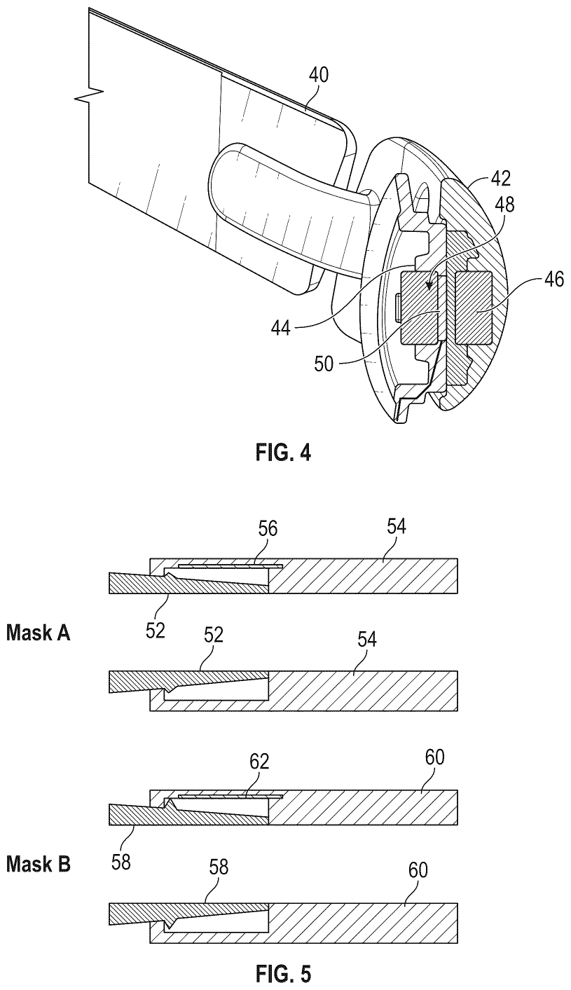

Secondly, the force sensors can be accurate enough to measure only small differences in attractive force, so there are many discrete intervals to identify different masks using this technique without hampering patient experience. This embodiment is illustrated in FIG. 4, which includes a headgear component 40 having a headgear magnetic connector member 42 that is coupled to a mask magnetic connector member 44. Headgear magnetic connector member 42 has a magnet 46 housed therein, and mask magnetic connector member 44 has a magnet 48 housed therein. A force sensor 50 is provided between magnet 46 and magnet 48. Low-cost force sensors are available that are extremely flat (<0.25 mm) to enable them to be positioned between the two magnets in order to directly measure the force between the magnets after a connection is made. The magnets can have specific field strengths to differentiate different types of masks.

In the embodiments described this far, static force measurements are used to identify the patient interface device component as described. As an alternative, dynamic force measurement during connection of the mask may also be used, Several of such embodiments are described below.

In one dynamic force based embodiment of the disclosed concept. the force or force change is measured during the connection of the mask to the connecting element (such as the tubing). The force profile in time during the attachment of the mask is indicative to the type of mask that is being attached. The force profile may be influenced by the mechanical geometry of the mask attachment and may be influenced by specific differences in mechanical features that cause a change in force profile during attachment of the mask to the machine (for instance via the tubing). An example of this is alternative embodiment, which may be used in connection with pressure generating device 4 in system 2, is shown in FIG. 5 for differentiating between mask A and mask B. In this embodiment, mask A includes a mask connector 52 structured to be coupled to a delivery conduit by way of a coupling 54 having a retainer clip, wherein coupling 54 has a force sensor 56 in the form of a complaint force sensor, mounted therein.

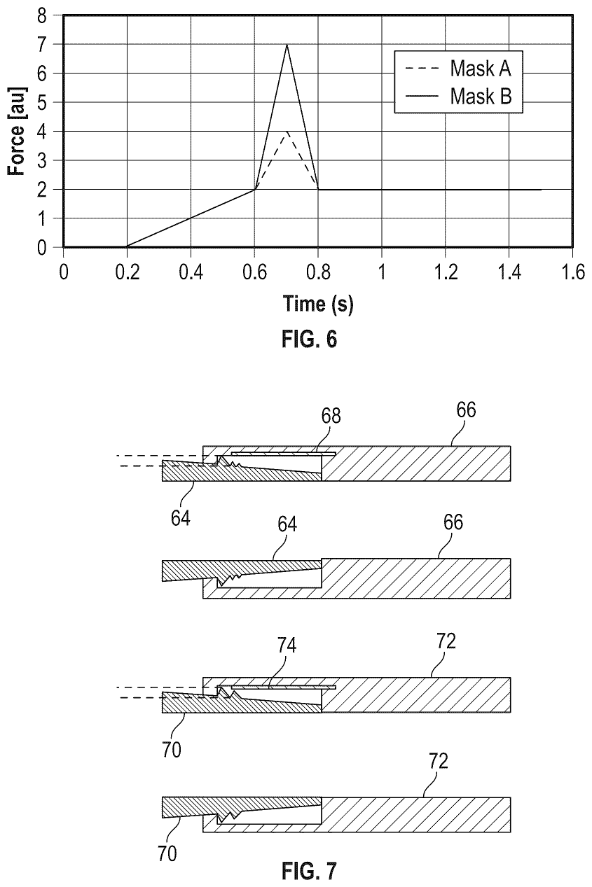

Mask B includes a mask connector 58 structured to be coupled to a delivery conduit by way of a coupling 60 having a retainer clip, wherein coupling 60 has a force sensor 62 in the form of a complaint force sensor, mounted therein. As seen in FIG. 5, the clip engagement geometry in connectors 52 and 58 differs (the clip engaging member in connector 52 is smaller than the clip engaging member in connector 58) such that connector 52 will deform the clips of coupling 54 less than connector 58 will deform the clips of coupling 60 during the connection process. While the final attachment force may be similar between mask A and mask B, the profile during attachment will be different for the two mask types. By registering the maximum force during attachment, or the force change (derivative) over time, the different masks can be identified according to this embodiment. The advantage of this method is that while the final connection is the same for both types of mask, the maximum force needed to overcome the larger retainer elements during connection is different and can be used for identifying the mask type. This profile difference is illustrated in FIG. 6. In this embodiment, however, the sampling rate of the force sensor may need to be higher than in the static forced based embodiments described herein to avoid missing the maximum force.

In the above example, the maximum connecting and disconnecting force is different for each mask. Although, as explained elsewhere herein, there is ample window for slight variations in connection forces between mask types, the connection element can also be designed in such a way that the force profile is different but the maximum force that the patient needs to exert is always the same for all mask types. In this way, connecting different mask will not feel any different for the patient but the force profile will be different for each specific mask so that the masks can still be identified. This can be realized by making a signature profile for each connection by different mechanical elements, but having a similar final retention element for all masks. An example of this is alternative embodiment, which may be used in connection with pressure generating device 4 in system 2, is shown in FIG. 7 for differentiating between mask A and mask B. In this embodiment, mask A includes a mask connector 64 structured to be coupled to a delivery conduit by way of a coupling 66 having a retainer clip, wherein coupling 66 has a force sensor 68 in the form of a complaint force sensor, mounted therein.

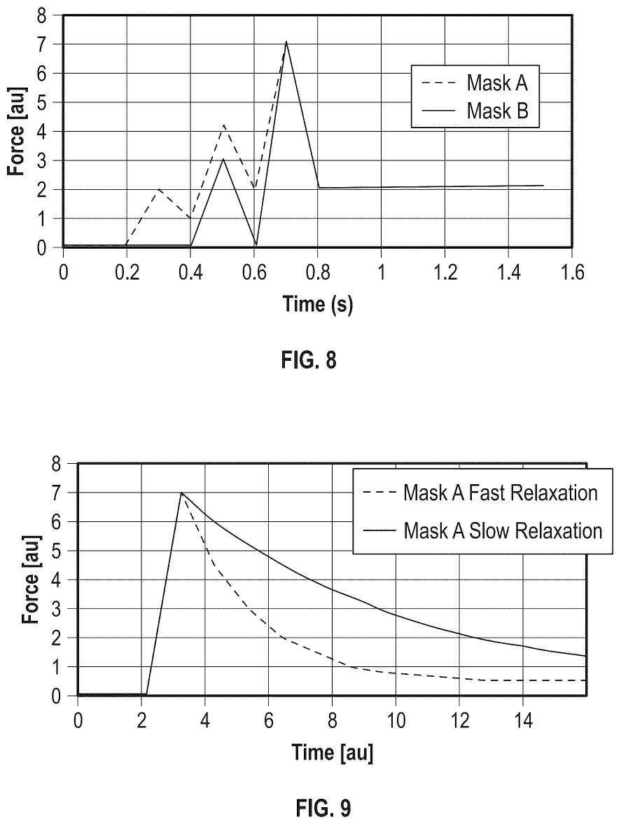

Mask B includes a mask connector 70 structured to be coupled to a delivery conduit by way of a coupling 72 having a retainer clip, wherein coupling 72 has a force sensor 74 in the form of a complaint force sensor, mounted therein. As seen in FIG. 7, the clip engagement geometry in connectors 64 and 70 differs such that connector 64 will deform the clips of coupling 66 less than connector 70 will deform the clips of coupling 72 during the connection process. In this embodiment, as shown in FIG. 8, the profile during attachment will be different for the two mask types, but the maximum force that the patient needs to exert is the same for the two mask types.

In yet a further alternative embodiment, the mechanical design of the mask connector and coupling can be similar between two mask A and B, but with different materials with different properties being used between Mask A and Mask B. Such differences will cause differences in the dynamic force profiles of the connections as illustrated in FIG. 9. For example, using materials with different viscoelastic properties in the connector for each mask will result in initially the same connection force but different decay times. This embodiment has an advantage that the attachment force is the same for each mask when attaching (i.e. the patient feels no difference) and a high sampling rate is not needed.

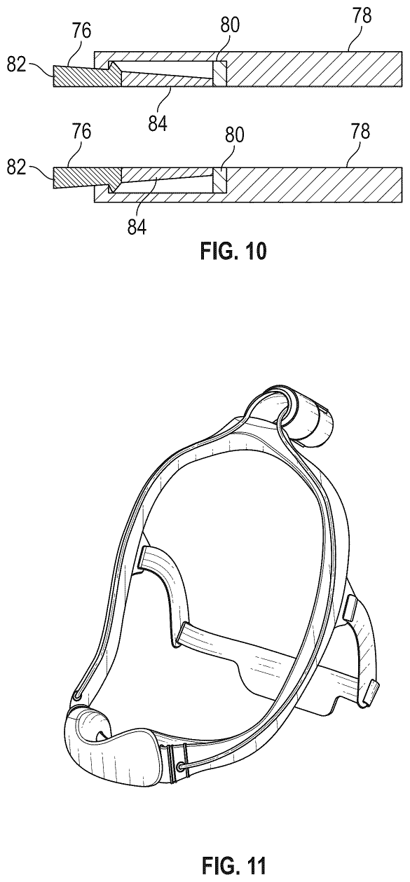

Another alternative embodiment is shown in FIG. 10, where a mask includes a mask connector 76 that is structured to be coupled to a delivery conduit 78 (or a fluid coupling conduit), wherein delivery conduit 78 has a force sensor 80 in the form of a capacitive compression sensor, mounted therein. As illustrated in FIG. 10, mask connector 76 has a first portion 82 made of a rigid material and a second portion 84 made of a viscoelastic material. In the embodiment, the viscoelastic element (second portion 84) causes a predetermined decay in the force on the force sensor, and can be designed such that the retaining force (the attachment force of the mask) always stays the same but the force on the force sensor itself is distinctly different for each mask depending on the material used for second portion 84 (i.e., the difference in viscoelastic properties define the characteristic force decay curve which can identify the type of mask being used).

In a further alternative embodiment, the weight of a mask cushion is measured through force sensors while the mask cushion is connected to a connecting element such as tubing or a headgear. The weight of the mask cushion as measured through force sensors is indicative to the type of mask that is being attached, as each mask cushion has its own weight. Since the force profile may be influenced by the relative position of the mask cushion towards the rest of the patient interface, a preferred positioning/orientation of the mask cushion and the rest of the patient interface during measurements (i.e., during a manual weighing step before the mask cushion is donned by the patient) should be prescribed. For example, it may be indicated that the mask should be held in such a way that the Fz is directed vertically while force measurements are made. FIG. 11 illustrates one particular way in which this embodiment may be implemented wherein force sensors are incorporated at the outer edges of the mask cushion (i.e., where the mask cushion connects to the tubing and/or headgear). In a further alternative implementation, the mask cushion may be fitted with an additional orientation sensor (such as an IMU sensor) such that the measured forces can be corrected for the (known) orientation of the mask during use (for instance directly after the patient puts the mask on), which eliminates the need for a manual weighing step at a predetermined orientation as described above. While weight sensors used for this embodiment as described thus far are force related, other weight sensors, like strain gauges, springs or even loadcells, may also be used. As noted, strain sensors modules may be attached to the headgear where it attaches to the mask cushion, or, alternatively, may be integrated in silicone tubing using piezoresistive fabric threads, sewn-in/molded in strain gages, or force sensors or strain gages integrated in the clips or molded in the silicone.

### Vibration-Based Embodiments

In one embodiment of this aspect of the disclosed concept, a set of connectors making a mechanical connection are designed to give a characteristic vibration when making the connection. The vibration is registered by a vibration sensor that is located close to one or both of the connectors to record the vibrations which occur when the connection is being made. The vibration profile is unique for each different mask type, making it possible to identify which mask is being connected to the therapy device. The identification can occur during attachment and/or detachment of the mask or mask element (cushion, straps, headgear parts, inserts etc.). In this embodiment, the connection geometry is designed in such a way that the vibration is present, with its characteristic profile, every time the connection is made, regardless of how the patient attaches the element. Examples of such a connection type include mechanical connections which operate using a build-up of potential energy, followed by a release of this energy (such as a snap-fit, push-through or bistable connection), or which use a magnetic snap connection utilizing one or more magnets that generate a controlled attractive force during the connection.

The vibration profile can be characterized by a specific resonance frequency, (initial) amplitude, or decay (damping) parameter, or a combination of these characteristics. These characteristics can be influenced by the design of the connectors, utilizing for instance: (i) specific geometric elements with characteristic mass and stiffness, (ii) materials with specific damping characteristics, or (iii) the input energy of the connection (defined by, for instance, magnet strength or mechanical energy release). Sensors capable of recording mechanical vibrations for use in this aspect of the disclosed concept include standard vibration sensors such as, without limitation, accelerometers, piezoelectric sensors or piezoresistive sensors.

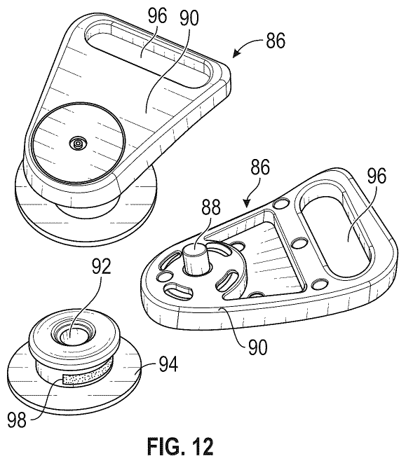

FIG. 12 shows one exemplary implementation of this embodiment. As seen in FIG. 12, a headgear clip 86 is provided for attaching a headgear to a mask by a snap fit. Specifically, a peg 88 of a first clip member 88 is structured to be received in a hole 92 of a second clip member 94. Second clip member 94 is structured to be attached to a mask frame or cushion. First clip member 90 includes a loop portion 96 for receiving a headgear strap so that the headgear can be releasably coupled to the mask frame or cushion. In addition, second clip member 94 includes a vibration sensor 98 for measuring the vibration profile when peg 88 is received in hole 92.

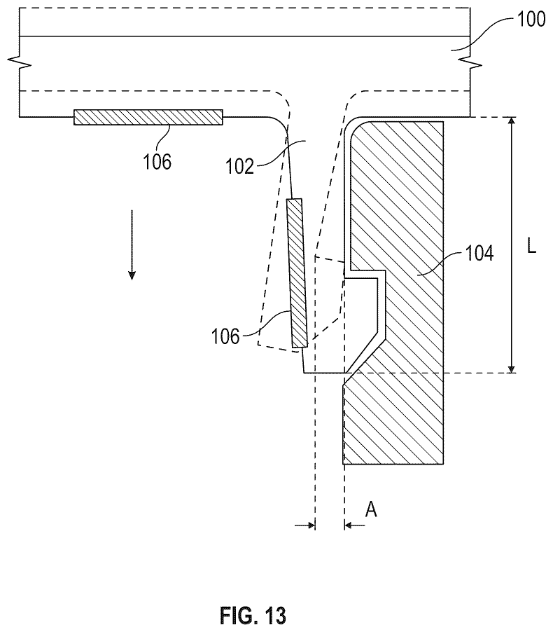

FIG. 13 shows another exemplary embodiment of this aspect of the disclosed concept that may be used in connection with pressure generating device 4 in system 2 of FIG. 1. As seen in FIG. 13, this embodiment employs a connector clip of a snap-fit connection type. In particular, this embodiment includes a clip member 100 that includes a cantilever beam 102 for making a snap fit connection to a component 104 as shown. Cantilever beam 102 includes a number of vibration sensors 106 attached thereto. The mechanical properties of this connection may be approximated using a model of a damped cantilever beam structure with a first frequency of approximately:

$${f}_{1} = \frac{{( 1.875 )}^{2} {( 1 - {ζ}^{2} )}^{0.5} {( \frac{EI}{{mL}^{4}} )}^{0.5}}{2 π}$$

where ζ is the damping factor of the connector, EI is the beam stiffness, m is the mass and L is the length of the beam. If, for instance, clip member 100 is made of standard rigid plastic material (e.g., nylon or polycarbonate) that is 1 cm long and has a cross section of 2 mm×3 mm, the 1^{st} resonance frequency will be around 65 kHz. This frequency is easily measurable with vibration sensor(s) 106, such as piezoelectric sensors.

Typically, a small number of oscillations are needed to reliably measure a frequency, for instance by using FFT to analyse the signal in the frequency domain. This can be realized by design of the snap-fit connector by leaving enough space for the connector to resonate shortly after being pushed in place. Furthermore, in order to distinguish between different masks, the resonance frequency may be influenced by altering the design of the snap-fit connection, for instance by changing the effective length of the beam (L) in discrete steps. By increasing the length of the connector by 0.5 mm, the characteristic frequency will decrease to about 57 kHz, while decreasing the length by 0.5 mm will increase the frequency to 73 kHz. These characteristic frequencies can be used to distinguish small, medium and large cushions or provide identification information on different types of masks. Alternatively, the starting amplitude, A, can be influenced by the design of the snap-fit connector to provide information on the type of mask being connected. Additionally, the materials used can influence the damping factor ζ, which influences the decay of the vibration. In essence, any unique combination of these characteristics can be identified by a vibration sensor such as vibration sensor(s) 106.

Vibration sensor(s) 106 may be powered by a power source in the mask (battery) or power may be provided from the therapy device (e.g., pressure generating device 4). The communication of data may be wireless or wired (to the therapy device). In this way, the snap connector of this embodiment can be designed in such a way that the vibration characteristic defines the type and size of the mask cushion, such that the mask type and size can be verified each time the patient puts on the mask before starting a therapy session. In practice, by way of example, a vibration sensor located in the headgear clips and powered via an electrical connection through the tubing may measure the vibration each time the patient attaches their mask clips.

In another embodiment, the vibration is measured by the change in magnetic field at the sensor location due to the snap-on connection action. A time varying magnetic field may be measured by an inductor, such as a wire coil, where the voltage is a function of the coil characteristics and the time variation of the magnetic field. An industry standard surface mount 22 uH coil with dimensions 2.5 mm×2.5 mm×2 mm, (costing less than 0.09 EUR) is capable of measuring around 5-10 mV peak, making it possible to sense a magnetic connection being made to the patient interface. This functionality may be achieved by the particular geometric arrangement of the snap-on connector, which determines, for a given magnetic strength, the dynamics of the snap-on connection, resulting in a signature voltage response in time. The voltage time response may be characterized by a certain geometric arrangement which may include structural design components as well as material properties which govern the mechanical characteristics of the snap-on connection. The snap-on connector of this embodiment may be designed in such a way that the connector can vibrate freely at the first resonance frequency (which is generally in the MHz range) for a substantial amount of time (enough to record and analyze the characteristics) before the snap connector sets into its fastened state. For instance, a design incorporating a beam with a specified resonance frequency may be used to identify a mask cushion when it is being snapped-on. The mass of the counter magnet that is being snapped on creates a vibration, which is registered by the coil.

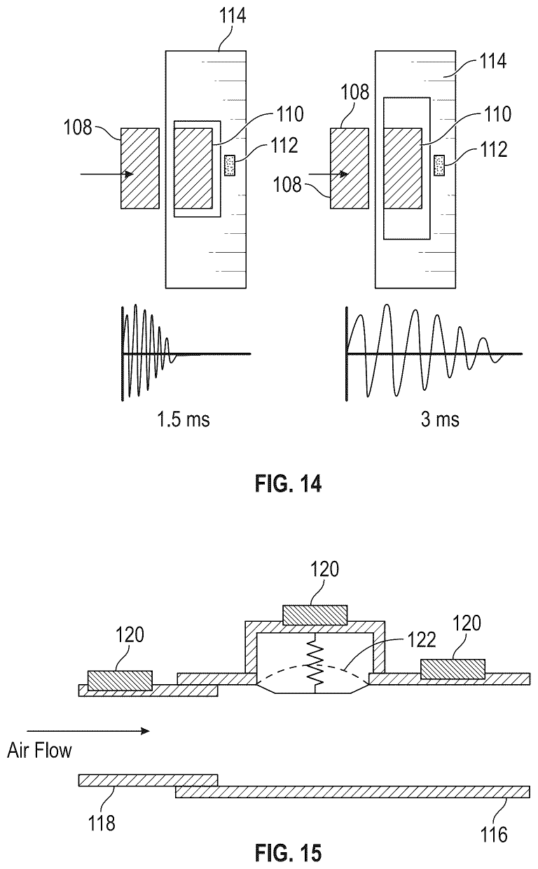

Altering the geometry or material of the beam will alter its vibration amplitude, resonance frequency and/or damping. By detecting the resonance frequency or the vibration pulse time, the type of mask can be identified. FIG. 14 illustrates an example geometry for implementing this embodiment where a magnetic connection is made using magnets 108 and 110 and an inductor-based sensor 112 (e.g., a coil) embedded in or attached to a connector structure. As shown in FIG. 14, different connector shapes will result in different response signatures (magnetic field changes) during connection to enable different connector shapes, and thus different connected masks, to be distinguished and identified.

In yet another embodiment, a vibration element that is not part of a connection but that is a characteristic element that is activated during therapy, for instance when pressure is applied to the mask, is used to measure a characteristic vibration. The vibration element may be located in the mask or mask cushion and the sensor may be located close to the vibration element, but not necessarily on the same part of the mask. For instance, the vibration sensor may be located on the tubing close to the mask connection and the vibration element may be located on the mask connector. When the vibration element is activated, the vibration travels through the connection and is recorded by the vibration sensor. As an example, such a vibration element can be a (preloaded) bi-stable element that is activated by a certain pressure. When activated, the element “pops” into its other stable position. At activation, the element vibrates with a specified frequency (determined by the structural design and materials used) until the vibration damps out naturally. When the pressure is released, the element always pops back to its original position due to the preloading spring.

FIG. 15 illustrates an example geometry for implementing this embodiment that includes a conduit 116, a tube connector 118, a number of vibration sensors 120, and a bistable element 122 (including a spring), wherein the vibration sensors are connected to the conduit 116, the tube connector 118 and/or the bistable element 122. Bistable element 122 is designed in such a way that: (i) the spring force (Fpr) is higher than the activation threshold of bistable element 122, and (ii) the force on bistable element 122 at the minimum therapeutic pressure (or any other specific pressure) is higher than the sum of the spring force and the activation threshold of bistable element 122. As a result, any application of therapy will result in an activation of bistable element 122 for the duration of the therapy, after which bistable element 122 will return to its initial state (aided by the spring). In this embodiment, the structural characteristics, and therefore the characteristics of the vibration that is caused by the activation of the bistable element 122, are unique for each mask type and can be used to identify which mask is connected to the therapy device.

In addition, each of these vibration-based embodiments may be used in connection with system 2 of FIG. 1 including pressure generating device 4 and electronics module 12. In such embodiments, controller 20 of electronics module 12 implements a vibration analysis module that is configured to determine the type and/or size of a component based on the sensor value(s) received from a vibration sensor. In one implementation, the type and/or size of the component of a patient interface device may be determined based on the sensor value(s) received from the vibration sensor using a comparison algorithm that is based on a lookup table or database of vibration values and corresponding patient interface device component types and sizes. In an alternative embodiment, the type and/or size of the component may be determined based on the sensor value(s) received from the vibration sensor using a trained machine learning system, such as a trained convolutional neural network (CNN), implemented by the vibration analysis module.

In the various embodiments described thus far, force or vibration data is used for determining the brand, type and/or size of the mask portion of a patient interface device. In a further embodiment, the combination of force and vibration data is used for determining the brand, type and/or size of the mask portion of a patient interface device. In this embodiment, the combination of the two signatures (force and vibration) results in a higher resolution detection system or an increased range (more different masks can be identified). This can be done by a combination of 2 sensors or a combined force/vibration sensor that can sense both the force and vibration. For instance, sensor 106 in FIG. 13 could have this functionality, where the both the vibration characteristics can be measured (as described in the text above) as well as the residual force after connection can be measured (i.e. the static end situation that is not related to the vibration characteristics of the latch element 102). This end force can be non-zero if the latch is designed such that it does not fully relax when in connected position, for instance if the cavity in element 104 is not as deep as it is currently drawn. Both force and vibration are independently variable by design so they can be used in combination according to this embodiment.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A pressure support system, comprising:
a pressure generating device for generating a flow of breathing gas;
a patient interface device for delivering the flow of breathing gas to the airways of a patient, wherein the patient interface device includes a mask portion, a headgear portion, and a conduit portion, the conduit portion being for receiving the flow of breathing gas from the pressure generating device;
an identification sensor arrangement coupled to the patient interface device, wherein the identification sensor arrangement includes:
(i) a force sensor structured and configured to generate force data indicative of a force generated during connection of the mask portion to the headgear portion or to the conduit portion or during use of the pressure support system, and/or
(ii) a vibration sensor structured and configured to generate vibration data indicative of vibrations generated during connection of the mask portion to the headgear portion or to the conduit portion or during use of the pressure support system; and
a controller structured and configured to receive an output of the identification sensor arrangement and determine a brand, type, and/or size of the mask portion based on the force data and/or the vibration data.

2. The pressure support system according to claim 1, wherein the force data is static force data or dynamic force data comprising a force profile over time, and wherein the vibration data is static vibration data or dynamic vibration data comprising a vibration profile over time.

3. The pressure support system according to claim 1, wherein the identification sensor arrangement includes the force sensor, wherein the patient interface device includes a clip connected to one of the mask portion, the headgear portion or the conduit portion, wherein the force sensor is directly connected to the clip or positioned between the clip and the one of the mask portion, the headgear portion or the conduit portion, and wherein the controller is structured and configured to determine the brand, type and/or size of the mask portion based on the force data.

4. The pressure support system according to claim 1, wherein the identification sensor arrangement includes the force sensor, wherein the mask portion includes a first magnetic connector member having a first magnet and the headgear portion or the conduit portion includes a second magnetic connector member having a second magnet, wherein the force data is indicative of a magnetic field strength between the first magnet and the second magnet, and wherein the controller is structured and configured to determine the brand, type and/or size of the mask portion based on the force data.

5. The pressure support system according to claim 1, wherein the identification sensor arrangement includes the force sensor directly connected to the conduit portion, wherein the conduit portion includes a retainer clip, wherein the patient interface device includes a connector structured to be received within the conduit portion and engage the retainer clip, wherein the force data is a force profile generated when the connector is received within the conduit portion, and wherein the controller is structured and configured to determine the brand, type and/or size of the mask portion based on the force data.

6. The pressure support system according to claim 1, wherein the identification sensor arrangement includes the force sensor comprising a capacitive compression sensor provided within the conduit portion, wherein the conduit portion includes a retainer clip, wherein the patient interface device includes a connector structured to be received within the conduit portion and engage the retainer clip, wherein the connector includes a first portion made of a rigid material and a second portion made of a viscoelastic material, wherein the force data is a force profile generated when the connector is received within the conduit portion and the viscoelastic material engages the capacitive compression sensor, and wherein the controller is structured and configured to determine the brand, type and/or size of the mask portion based on the force data.

7. The pressure support system according to claim 1, wherein the identification sensor arrangement includes the vibration sensor, wherein the patient interface device includes a connector assembly for connecting the mask portion to the headgear portion, wherein the vibration sensor is directly connected to the connector, and wherein the controller is structured and configured to determine the brand, type and/or size of the mask portion based on the vibration data.

8. The pressure support system according to claim 1, wherein the identification sensor arrangement includes the vibration sensor, wherein the patient interface device includes a connector assembly for connecting the mask portion to the headgear portion or the conduit portion, wherein the connector assembly includes a cantilever beam for making a snap fit connection, wherein the vibration sensor is directly connected to the cantilever beam, and wherein the controller is structured and configured to determine the brand, type and/or size of the mask portion based on the vibration data.

9. The pressure support system according to claim 1, wherein the identification sensor arrangement includes the vibration sensor comprising an inductor, wherein the mask portion includes a first magnetic connector member having a first magnet and the headgear portion or the conduit portion includes a second magnetic connector member having a second magnet, wherein the vibration data is indicative of a change in magnetic field over time responsive to the first magnetic connector member being connected to the second magnetic connector member, and wherein the controller is structured and configured to determine the brand, type and/or size of the mask portion based on the vibration data.

10. A method for automatically identifying a mask used in a pressure support system including a pressure generating device for generating a flow of breathing gas, a patient interface device for delivering the flow of breathing gas to the airways of a patient, wherein the patient interface device includes a mask portion, a headgear portion, and a conduit portion, the conduit portion being for receiving the flow of breathing gas from the pressure generating device, comprising;
receiving in a controller an output of an identification sensor arrangement coupled to the patient interface device, wherein the identification sensor arrangement includes:
(i) a force sensor structured and configured to generate force data indicative of a force generated during connection of the mask portion to the headgear portion or to the conduit portion or during use of the pressure support system, and/or
(ii) a vibration sensor structured and configured to generate vibration data indicative of vibrations generated during connection of the mask portion to the headgear portion or to the conduit portion or during use of the pressure support system; and
determining in the controller a brand, type and/or size of the mask portion based on the force data and/or the vibration data.

11. The method according to claim 10, wherein the force data is static force data or dynamic force data comprising a force profile over time, and wherein the vibration data is static vibration data or dynamic vibration data comprising a vibration profile over time.

12. The method according to claim 10, wherein the identification sensor arrangement includes the force sensor, wherein the patient interface device includes a clip connected to one of the mask portion, the headgear portion or the conduit portion, wherein the force sensor is directly connected to the clip or positioned between the clip and the one of the mask portion, the headgear portion or the conduit portion, and wherein the controller determines the brand, type and/or size of the mask portion based on the force data.

13. The method according to claim 10, wherein the identification sensor arrangement includes the force sensor, wherein the mask portion includes a first magnetic connector member having a first magnet and the headgear portion or the conduit portion includes a second magnetic connector member having a second magnet, wherein the force data is indicative of a magnetic field strength between the first magnet and the second magnet, and wherein the controller determines the brand, type and/or size of the mask portion based on the force data.

14. The method according to claim 10, wherein the identification sensor arrangement includes the force sensor directly connected to the conduit portion, wherein the conduit portion includes a retainer clip, wherein the patient interface device includes a connector structured to be received within the conduit portion and engage the retainer clip, wherein the force data is a force profile generated when the connector is received within the conduit portion, and wherein the controller determines the brand, type and/or size of the mask portion based on the force data.

15. The method according to claim 10, wherein the identification sensor arrangement includes the force sensor comprising a capacitive compression sensor provided within the conduit portion, wherein the conduit portion includes a retainer clip, wherein the patient interface device includes a connector structured to be received within the conduit portion and engage the retainer clip, wherein the connector includes a first portion made of a rigid material and a second portion made of a viscoelastic material, wherein the force data is a force profile generated when the connector is received within the conduit portion and the viscoelastic material engages the capacitive compression sensor, and wherein the controller determines the brand, type and/or size of the mask portion based on the force data.

16. The method according to claim 10, wherein the identification sensor arrangement includes the vibration sensor, wherein the patient interface device includes a connector assembly for connecting the mask portion to the headgear portion, wherein the vibration sensor is directly connected to the connector, and wherein the controller determines the brand, type and/or size of the mask portion based on the vibration data.

17. The method according to claim 10, wherein the identification sensor arrangement includes the vibration sensor, wherein the patient interface device includes a connector assembly for connecting the mask portion to the headgear portion or the conduit portion, wherein the connector assembly includes a cantilever beam for making a snap fit connection, wherein the vibration sensor is directly connected to the cantilever beam, and wherein the controller determines the brand, type and/or size of the mask portion based on the vibration data.

18. The method according to claim 10, wherein the identification sensor arrangement includes the vibration sensor comprising an inductor, wherein the mask portion includes a first magnetic connector member having a first magnet and the headgear portion or the conduit portion includes a second magnetic connector member having a second magnet, wherein the vibration data is indicative of a change in magnetic field over time responsive to the first magnetic connector member being connected to the second magnetic connector member, and wherein the controller determines the brand, type and/or size of the mask portion based on the vibration data.

19. The method according to claim 10, wherein the identification sensor arrangement includes the vibration sensor, wherein the patient interface device includes a bistable element including a spring, wherein the bistable element is structured to move from an initial state to an activated state in in response to the flow of breathing gas, wherein in the activation state the bistable element will vibrate, wherein the vibration data is indicative of the vibration of the bistable element, and wherein the controller determines the brand, type and/or size of the mask portion based on the vibration data.

20. The method according to claim 11, wherein the receiving includes receiving in the controller an output of an identification sensor arrangement coupled to the patient interface device, wherein the identification sensor arrangement includes: (i) a force sensor structured and configured to generate force data indicative of a force generated during connection of the mask portion to the headgear portion or to the conduit portion or during use of the pressure support system, and (ii) a vibration sensor structured and configured to generate vibration data indicative of vibrations generated during connection of the mask portion to the headgear portion or to the conduit portion or during use of the pressure support system, and wherein the determining includes determining in the controller a brand, type and/or size of the mask portion based on the force data and the vibration data.
